(19) United States
(12) Patent Application Publication
(10) Pub. No.: US 2009/0124890 A1
(43) Pub. Date: May 14, 2009

(54) Method and a System for Assisting Guidance of a Tool for Medical Use

(76) Inventor: Raymond Derycke, Paris (FR)

Correspondence Address: YOUNG & THOMPSON, 209 Madison Street, Suite 500, ALEXANDRIA, VA, 22314, US

(21) Appl. No.: 11/884,577

(22) PCT Filed: Feb. 6, 2006
(86) PCT No.: PCT/FR2006/000262

§ 371 Date: Aug. 17, 2007

(30) Foreign Application Priority Data
Feb. 18, 2005 (FR) 0501750

(51) Int. Cl. A61B 5/05

(52) U.S. Cl. 600/424

(57) ABSTRACT

A system for assisting guidance of a tool manipulated by a practitioner relative to a portion of the human body. The system includes elements for making a three-dimensional first image of a first scene including the human body portion and a first reference associated therewith, elements for making a three-dimensional second image of a second scene including the tool and a second reference associated therewith, the second image being made relative to the first reference, elements for making a third image constituted by superposing the first and second images so that the representations of the first reference contained respectively in the first and second images substantially coincide, and elements for projecting the third image into the field of view of the practitioner's eye while manipulating the tool.

The present invention relates to methods and systems for assisting guidance of a tool suitable for being manipulated by a practitioner or the like relative to a portion of the human body that comprises at least a portion of bone covered in flesh, the invention finding particularly, but not exclusively, an application that is particularly advantageous in fitting a dental implant in a jaw.

Some practitioners need to perform surgical type interventions or the like, e.g. in order to put implants into place. This applies for example to implanting prostheses or the like in the orthopedic, ear-nose-throat (ENT), dental, etc. . . . fields.

For this purpose, a practitioner is almost always obliged to drill into bony portions in order to place the rods of the implant therein. It can readily be understood that drilling bony portions in this way, e.g. vertebrae or jaw bones, is relatively difficult since it is absolutely essential to ensure that the bit of the drilling tool does not touch any sensitive elements such as the spinal cord for vertebrae, the ophthalmic nerve in the skull, or the dental nerve in a jaw.

In order to perform such difficult operations, confidence is generally put in the experience of the practitioner, however such experience alone cannot solve certain problems.

To solve this problem, methods and devices have been developed for assisting guidance of a tool that is suitable for being manipulated by a practitioner such as a surgeon, dentist, or the like, relative to a human body portion or the like.

One such method is described for example in U.S. Pat. No. 6,006,126. The method described in that US document relates to a method of recording data representing a stereographic image, in particular in the medical field. It consists essentially in making first images of a portion of a human body or the like using a scanner that gives a plurality of plane images, i.e. images in two dimensions only, and second images that are stereographic, i.e. in three dimensions, using video cameras, and then in combining the first and second images.

Nevertheless, guiding the tool with the help of that method is possible only under the express condition of the human body being stationary and not moving at all, i.e. not even subject to any parasitic displacements.

Such a method and the device implementing it are therefore limited in their applications and can even be dangerous if the portion of the human body on which the tool is to act is not held perfectly still.

The present invention thus seeks to implement a method and to provide a system for assisting guidance of a tool suitable for being manipulated by a practitioner of the surgeon, dentist, or like type, relative to a human body portion comprising at least a portion of bone covered in flesh, without it being necessary to keep said human body portion stationary, the method and system finding particularly advantageous applications in numerous fields, in particular in the field of dentistry where it is practically impossible to keep the head of the patient absolutely stationary.

More precisely, the present invention provides a method of assisting the guidance of a tool suitable for being manipulated by a practitioner or the like relative to a portion of the human body, said human body portion comprising at least a portion of bone covered in flesh, the method being characterized by the fact that it consists:
in making a three-dimensional first image of a first scene comprising the human body portion and a first reference associated with said human body portion;
in making a three-dimensional second image of a second scene comprising said tool and a second reference associated with said tool, said second image being made relative to said first reference;
in making a third image constituted by superposing said first and second images so that the representations of the first reference contained respectively in the first and second images coincide substantially; and
in projecting the third image into the field of view of an eye of the practitioner while ready to manipulate said tool.

The present invention also provides a system for assisting the guidance of a tool suitable for being manipulated by a practitioner or the like, relative to a portion of the human body comprising at least a portion of bone covered in flesh, characterized by the fact that it comprises:
means for taking a three-dimensional first image of a first scene comprising the human body portion and a first reference associated with said portion;
means for making a three-dimensional second image of a second scene comprising said tool and a second reference associated with said tool, said second image being made relative to said first reference;
means for making a third image constituted by superposing said first and second images so that the representations of the first reference contained respectively in the first and second images coincide substantially; and
means for projecting said third image into the field of view of an eye of the practitioner while ready to manipulate said tool.

Other characteristics and advantages of the invention appear from the following description given with reference to the accompanying drawings by way of non-limiting illustration, in which:

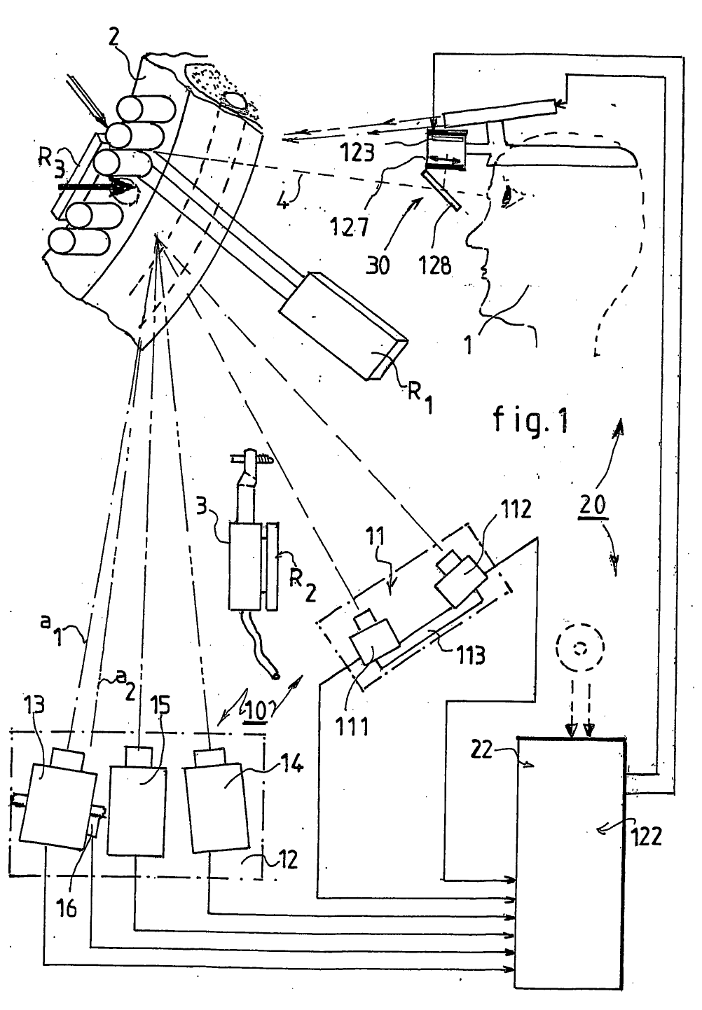

FIG. 1 is a block diagram showing the principles of an embodiment of the system of the invention, in an application to the field of dentistry; and

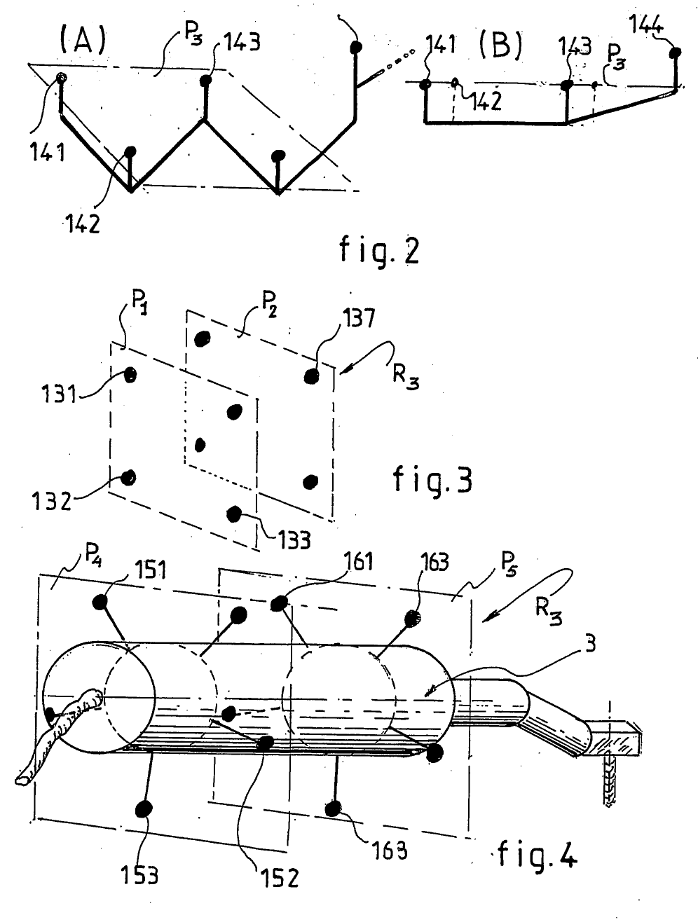

FIGS. 2 to 4 show three respective possible industrial embodiments of three of the elements shown diagrammatically in FIG. 1.

It is stated initially that in the figures, the same references are used to designate the same elements, regardless of the figure in which they appear and regardless of the form in which the elements are shown.

Similarly, if elements are not specifically referenced in one of the figures, their references can easily be found by referring to another figure.

It is also stated that the figures show essentially a single embodiment of the subject matter of the invention, but that other embodiments can exist that satisfy the definition of the invention.

The method and the system of the invention are described below with reference to a particular example of a dental practitioner putting a dental implant into place. However, as specified in the introduction of the present description, the present invention can naturally also be implemented in many other medical fields, such as, for example the orthopedic and ENT fields.

Implementing the method serves to assist in guiding a tool 3, e.g. a drill or the like, that is suitable for being handled by a practitioner 1 of the dental type relative to a portion of the human body that includes at least a portion of bone covered in flesh, i.e. in the present example a portion of jaw 2 together with the corresponding teeth.

The method of the invention consists initially in making a three-dimensional first image of a first scene comprising the portion of the human body, specifically the jaw 2, and a first reference R₁ associated with the jaw 2, and then in making a three-dimensional second image of a second scene comprising the tool 3, e.g. a drill or the like, and a second reference R₂ associated with the tool, the second image being made relative to the first reference R₁, then in making a third image constituted by superposing the first and second images so that the representations of the first reference R₁ contained respectively in each of the first and second images substantially coincide, and advantageously coincide completely with the greatest possible accuracy, and finally in projecting said third image into the field of view 4 of an eye of the practitioner, specifically the dentist 1, while ready to manipulate the tool 3.

In this way, at all times, the dentist can know exactly where the tool 3 is situated relative to the patient's jaw portion 2, firstly by seeing the third image, and secondly by looking directly in his or her own field of view while it includes the jaw portion.

However, in a particularly advantageous implementation of the method, in order to make the first image, a fourth image is made of the first scene by stereovision 11, and then a three-dimensional fifth image is made of the same above-defined first scene, i.e. the scene comprising the jaw portion 2 and the first reference R₁, by using at least one of the following techniques: X-ray radiography 13; raster analysis 14 by radiation that is absorbed relatively little by flesh and that is suitable for being reflected by the portion of bone; and X-ray tomographic scanning 15; and then superposing said fourth and fifth images so as to make an image constituting the first image as defined above.

When the fifth image is to be obtained by X-ray radiography 13, it is advantageous to make at least two two-dimensional images of the first scene and of a third reference R₃ at two respective different angles a₁, a₂, the third reference being constituted by radiographic markers that are opaque to X-rays and that are coupled to the jaw portion 2 in which the implant is to be placed, and in combining the at least two two-dimensional images so as to obtain in known manner the three-dimensional fifth image with reference to the reference R₃.

Advantageously, the third reference R₃ is constituted by markers substantially constituting points internal to the jaw portion 2, specifically portions of bone or bony anatomical points that are relatively opaque to X-rays and completely stationary relative to the jaw portion.

When the third reference R₃ is implemented by using bony points in the jaw portion 2 that are opaque to X-rays, it is advantageous to define the third reference by at least two planes intersecting on a straight line, the straight line and the two planes being determined by a plurality of said bony points.

In this way, by using the images obtained by implementing the above-defined method, it is possible to determine the position of the dental nerve canal relative to the first reference R₁, and thus for example to generate and emit a signal in any possible form for warning the dentist if the tool 3 being manipulated to prepare the orifice for receiving the implant accidentally risks passing through the canal and touching the dental nerve. The practitioner can then immediately rectify the orientation of the tool in order to drill along an optimal axis without danger for the patient. It is also possible to issue a signal for stopping the operation of the tool.

As defined above, in order to make the fifth image, the method may also consist in determining at least three points associated with the portion of bone by means of a depth probe, and in defining a reference surface relative to the first reference R₁ as a function of the at least three points associated with said portion of bone.

The radiation that is absorbed relatively little by flesh and that is suitable for being reflected by the portion of bone, and that is used for the process of raster analysis 14 may, for example, be one of the following kinds of radiation: ultrasound; coherent light as delivered by a laser generator; or the like.

As mentioned above, in its final step, the method consists in superposing the first and second images in the field of view of an eye of the practitioner. The method then consists advantageously in processing the first and second images so as to transform them into digital images and in superposing the two digital images so as to obtain a final digital image which is in turn converted into an optical image constituting the third image that can be seen by the dentist 1.

Under such circumstances, projecting the third image into the field of view 4 of an eye of the dentist 1 while ready to manipulate the tool 3 consists in focusing the third image outside the field of view 4, e.g. at a distance of about forty to fifty centimeters, and in delivering the image to the eye of the dentist 1 by partial reflection, e.g. by means of a semitransparent and semireflective plate.

To summarize, the method of the invention as described above in a particular example is characterized by the following points taken separately or in combination:

A) A method of assisting the guidance of a tool (3) suitable for being manipulated by a practitioner (1) or the like relative to a portion (2) of the human body, said human body portion comprising at least a portion of bone covered in flesh, the method being characterized by the fact that it consists:
in making a three-dimensional first image of a first scene comprising the human body portion (2) and a first reference (R₁) associated with said human body portion (2);
in making a three-dimensional second image of a second scene comprising said tool (3) and a second reference (R₂) associated with said tool, said second image being made relative to said first reference (R₁);
in making a third image constituted by superposing said first and second images so that the representations of the first reference (R₁) contained respectively in the first and second images coincide substantially; and
in projecting the third image into the field of view (4) of an eye of the practitioner (1) while ready to manipulate said tool (3).

B) A method according to point A), characterized by the fact that making the first image consists in making a fourth image of the first scene by stereovision (11), then in making a three-dimensional fifth image of the first scene by at least one of the following techniques: X-ray radiography (13); raster analysis (14) by radiation that is absorbed relatively little by flesh and that is suitable for being reflected by the portion of bone (14); and X-ray tomographic scanning (15); and in superposing the fourth and fifth images in order to make said first image.

C) A method according to point B), characterized by the fact that making the fifth image by X-ray radiography consists in making at least two two-dimensional images of the first scene and a third reference (R₃), under two respective different angles (a₁, a₂), said third reference (R₃) being constituted by radiographic markers that are opaque to X-rays and that are coupled to said human body portion (2) in which the implant is to be placed, and in combining said at least two two-dimensional images to obtain said three-dimensional fifth image.

D) A method according to point C), characterized by the fact that it consists in making the third reference (R₃) by implementing at least one of the following two processes: making substantially point references external to said human body portion (2) that are opaque to X-rays and associated in stationary manner with said human body portion (2); and using bony portions of said human body portion (2) that are opaque to X-rays.

E) A method according to point D), characterized by the fact that when the third reference is made by using bony portions of said human body portion (2) that are opaque to X-rays, it consists in defining said third reference by at least two planes that intersect along a straight line, said line and said two planes being determined by a plurality of bony points belonging to said human body portion (2).

F) A method according to any one of points C) to E), characterized by the fact that said human body portion is a jaw portion, the method consisting in determining in said fifth image the position of the dental nerve canal relative to the first reference (R₁).

G) A method according to point B), characterized by the fact that making the fifth image by raster analysis using radiation that is absorbed relatively little by flesh and that is suitable for being reflected by the portion of bone consists in determining at least three points associated with said portion of bone by means of a depth probe, and in defining a reference surface relative to the first reference (R₁) as a function of said at least three points associated with said portion of bone.

H) A method according to point G), characterized by the fact that the radiation that is absorbed relatively little by flesh and that is suitable for being reflected by the portion of bone used in raster analysis is one of the following radiations: ultrasound and coherent light.

I) A method according to any one of points A) to H), characterized by the fact that the superposition of said first and second images in the practitioner's field of view consists:
in processing said first and second images and in transforming them into a final digital image; and
in converting said final digital image into an optical image constituting said third image.

J) A method according to point I), characterized by the fact that the projection of the third image in the field of view (4) of an eye of the practitioner (1) while ready to manipulate said tool (3) consists in focusing said third image outside the field of view (4) and in delivering it into said field of view.

The invention also provides a system for implementing the above-defined method, said system being described below with reference to the figures accompanying the present description.

The diagrammatic figures show one possible and highly advantageous embodiment of the system of the invention for assisting in the guidance of a tool 3, such as a drill or the like, suitable for being manipulated by a practitioner such as a dentist 1 or the like, relative to a portion of the human body that includes at least a portion of bone covered in flesh, such as a jaw portion 2.

The system comprises means 10 for making a three-dimensional first image of a first scene comprising the human body portion 2 and a first reference R₁ associated with said portion, means 10 for making a three-dimensional second image of a second scene comprising the tool 3 and a second reference R₂ associated with the tool, the second image being made relative to the first reference R₁, means 20 for making a third image constituted by superposing the first and second images so that the representations of the first reference R₁ contained respectively in the first and second images coincides substantially, and means 30 for projecting said third image into the field of view 4 of an eye of the dental practitioner 1 while ready to manipulate the tool 3.

Embodiments of the means defined above by their function are described below.

In particular, the means for making the first image are advantageously constituted by means 11 for making a fourth image by stereovision of the above-defined first scene, means 12 for making a three-dimensional fifth image of the same first scene by at least one of the following techniques: X-ray radiography 13; raster analysis 14 using radiation that is absorbed relatively little by flesh and that is suitable for being reflected by the portion of bone; X-ray scanning tomography 15; and computer type means 22 for superposing the fourth and fifth images, said superposition constituting the first image.

In a preferred embodiment, the means 12 for making the three-dimensional fifth image of the first scene by X-ray radiography 13 comprise a third reference R₃, one embodiment of which is shown in FIG. 3, and is constituted by radiographic markers that are opaque to X-rays, means for coupling said third reference to the jaw portion 2, means 16 for making at least two two-dimensional images of the first scene and the third reference R₃ under two respective different angles a₁, a₂, and means 22 for combining the two two-dimensional images, this combination constituting the three-dimensional fifth image.

FIG. 3 shows an advantageous embodiment of the third reference R₃. It is constituted by at least six substantially point-like radiographic markers 131, 132, 133, . . . , 137 that are opaque to X-rays and that are disposed relative to one another in groups of three to define two substantially parallel planes P₁, P₂, e.g. on two superposed plates of a material that is transparent to X-rays.

The means 12 for making the fifth image by raster analysis 14 using radiation that is absorbed relatively little by flesh and that is suitable for being reflected by the portion of bone, such as the jaw portion 2, comprise means for determining at least three points associated with the bone portion, and means for defining relative to the first reference R₁, a three-dimensional reference surface as a function of said at least three points associated with the portion of bone.

These means for determining at least three points associated with the portion of bone advantageously comprise, for example: a depth probe suitable for emitting ultrasound or even coherent light radiation emitted by a generator of laser type or the like.

As mentioned above, the system of the invention includes a first reference R₁. This first reference R₁ is advantageously constituted, as shown in FIGS. 2A and 2B, by at least four points 141, 142, 143, 144 of brightness that is in contrast relative to the brightness of their surroundings, and advantageously opaque to X-rays, one of these four points 144 being situated outside the plane P₃ defined by the other three points 141, 142, 143. By way of example, these points are black metal beads disposed at the tops of respective uprights secured to a support rod of white plastics material or the like which itself may be secured to a healthy tooth of the jaw, for example, by means of a collar or the like, and including a clamping screw.

The second reference R₂ is advantageously constituted, as shown in FIG. 4, by a plurality of points 151, 152, 153, . . . , 161, 162, 163 of brightness that is in contrast relative to the brightness of their surroundings, these points being constituted, for example, by black metal beads mounted on support rods associated with the tool 3 so that at least three of these points are always visible to the dentist 1 and to the stereovision cameras, regardless of the position of the tool 3 relative to the first reference R₁.

In addition, and preferably, the points of contrasting brightness 151, 152, 153, . . . , 161, 162, 163 associated with the tool 3 are distributed around the tool in substantially regular manner and in two planes P₄ and P₅ that are substantially mutually parallel and do not coincide, each of these two planes P₄ and P₅ including at least three of these points.

As mentioned above, the system also includes means 20 for making the third image. They are advantageously constituted by processor means 122 of the computer type based on a microprocessor, suitable for processing the first and second images to transform them into digital images, thus making it easy by means of suitable software to superpose them to produce a final digital image, and means 123 for converting said final digital image into an optical image constituting the above-defined third image suitable for being seen by the dentist.

Under such circumstances, the means 30 for projecting the third image into the field of view 4 of an eye of the practitioner 1 ready to manipulate the tool 3, are advantageously constituted by means 127 for focusing the third image outside the field of view 4, and a semireflective plate 128 for causing the optical axis of the focusing means 127 to coincide with the axis of the field of view 4 so as to deliver the third image to the eye of the practitioner, i.e. the dentist 1 in the example selected for the present description.

Under such circumstances, it is advantageous for the means 11 for making the fourth image by stereovision to comprise two video cameras 111, 112, a transverse support 113, and means for mounting the two video cameras respectively substantially at the two ends of said support so that their optical axes coincide substantially on the jaw portion 2. The way in which such a stereovision device 11 operates is itself known and is not described more fully herein in order to simplify the present description.

The use of the system of the invention stems without any difficulty from the above description and from the description of the method given previously, and it is therefore not described in greater detail herein.

It is merely specified that while the dentist 1 is manipulating the tool 3 in the field of view 4, the dentist sees both: the tool directly, and as an image the entire scene with the bony portions of the jaw portion, the position of the tool, and more particularly of the drill bit relative to the bony portion, which would not be possible using direct vision only.

For example, as mentioned above, it is possible to determine and view the position of the dental nerve canal within the bony portion of the jaw, and to generate an alarm signal if the drill bit of the tool is oriented in such a manner as to run the risk of reaching the dental nerve canal, and even to generate a signal for stopping the operation of the tool 3.

It should also be emphasized that since the reference R₁ is associated with the patient, if the patient should move, as is always possible to a greater or lesser extent, it is possible continuously to reposition the first and second images so that the information given visually in the dentist's field of view 4 is always representative of reality regardless of the patient's position, naturally within given limits as is generally always the case.

With the above-described means, images are always repositioned with great accuracy, particularly when using ultrasound depth probe means for determining a plurality of reference points associated with the bony portion of the jaw, using the technique known in the art as “mapping”.

Naturally, the computer-type processor member 22 such as a computer with a microprocessor, operates in association with programmed software that comes within the competence of the person skilled in the art who can easily develop such software, in particular in the light of the above description of the method and the system of the invention.

1. A method of assisting the guidance of a tool (3) suitable for being manipulated by a practitioner (1) or the like relative to a portion (2) of the human body, said human body portion comprising at least a portion of bone covered in flesh, the method being characterized by the fact that it consists:
in making a three-dimensional first image of a first scene comprising the human body portion (2) and a first reference (R₁) associated with said human body portion (2);
in making a three-dimensional second image of a second scene comprising said tool (3) and a second reference (R₂) associated with said tool, said second image being made relative to said first reference (R₁);
in making a third image constituted by superposing said first and second images so that the representations of the first reference (R₁) contained respectively in the first and second images coincide substantially; and
in projecting the third image into the field of view (4) of an eye of the practitioner (1) while ready to manipulate said tool (3).

2. A method according to claim 1, characterized by the fact that making the first image consists in making a fourth image of the first scene by stereovision (11), then in making a three-dimensional fifth image of the first scene by at least one of the following techniques: X-ray radiography (13); raster analysis (14) by radiation that is absorbed relatively little by flesh and that is suitable for being reflected by the portion of bone (14); and X-ray tomographic scanning (15); and in superposing the fourth and fifth images in order to make said first image.

3. A method according to claim 2, characterized by the fact that making the fifth image by X-ray radiography consists in making at least two two-dimensional images of the first scene and a third reference (R₃), under two respective different angles (a₁, a₂), said third reference (R₃) being constituted by radiographic markers that are opaque to X-rays and that are coupled to said human body portion (2) in which the implant is to be placed, and in combining said at least two two-dimensional images to obtain said three-dimensional fifth image.

4. A method according to claim 3, characterized by the fact that it consists in making the third reference (R₃) by implementing at least one of the following two processes: making substantially point references external to said human body portion (2) that are opaque to X-rays and associated in stationary manner with said human body portion (2); and using bony portions of said human body portion (2) that are opaque to X-rays.

5. A method according to claim 4, characterized by the fact that when the third reference is made by using bony portions of said human body portion (2) that are opaque to X-rays, it consists in defining said third reference by at least two planes that intersect along a straight line, said line and said two planes being determined by a plurality of bony points belonging to said human body portion (2).

6. A method according to claim 3, characterized by the fact that said human body portion is a jaw portion, the method consisting in determining in said fifth image the position of the dental nerve canal relative to the first reference (R₁).

7. A method according to claim 2, characterized by the fact that making the fifth image by raster analysis using radiation that is absorbed relatively little by flesh and that is suitable for being reflected by the portion of bone consists in determining at least three points associated with said portion of bone by means of a depth probe, and in defining a reference surface relative to the first reference (R₁) as a function of said at least three points associated with said portion of bone.

8. A method according to claim 7, characterized by the fact that the radiation that is absorbed relatively little by flesh and that is suitable for being reflected by the portion of bone used in raster analysis is one of the following radiations: ultrasound and coherent light.

9. A method according to claim 1, characterized by the fact that the superposition of said first and second images in the practitioner's field of view consists:
in processing said first and second images and in transforming them into a final digital image; and
in converting said final digital image into an optical image constituting said third image.

10. A method according to claim 9, characterized by the fact that the projection of the third image in the field of view (4) of an eye of the practitioner (1) while ready to manipulate said tool (3) consists in focusing said third image outside the field of view (4) and in delivering it into said field of view.

11. A system for assisting the guidance of a tool (3) suitable for being manipulated by a practitioner (1) or the like, relative to a portion (2) of the human body comprising at least a portion of bone covered in flesh, according to claim 1, the system being characterized by the fact that it comprises:
means (10) for taking a three-dimensional first image of a first scene comprising the human body portion (2) and a first reference (R₁) associated with said portion (2);
means (10) for making a three-dimensional second image of a second scene comprising said tool (3) and a second reference (R₂) associated with said tool, said second image being made relative to said first reference (R₁);
means (20) for making a third image constituted by superposing said first and second images so that the representations of the first reference (R₁) contained respectively in the first and second images coincides substantially; and
means (30) for projecting said third image into the field of view (4) of an eye of the practitioner (1) while ready to manipulate said tool (3).

12. A system according to claim 11, characterized by the fact that the means (10) for making the first image are constituted by means (11) for making a fourth image of the first scene by stereovision, means (12) for making a three-dimensional fifth image of the same first scene by using at least one of the following techniques: X-ray radiography (13); raster analysis by radiation that is absorbed relatively little by flesh and that is suitable for being reflected by the portion of bone (14); and X-ray scanning tomography (15); and means (22) for superposing the fourth and fifth images, said superposition constituting said first image.

13. A system according to claim 12, characterized by the fact that the means (12) for making the three-dimensional fifth image of the first scene by X-ray radiography (13) comprise a third reference (R₃) (FIG. 3) constituted by radiographic markers that are opaque to X-rays, means for coupling said third reference to said human body portion (2), means (16) for making at least two two-dimensional images of the first scene and of the third reference (R₃), under two respective different angles (a₁, a₂), and means (22) for combining said at least two two-dimensional images, said combination constituting said three-dimensional fifth image.

14. A system according to claim 13, characterized by the fact that said third reference (R₃) (FIG. 3) is constituted by at least six substantially point-like radiographic markers (131, 132, 133, . . . , 137) that are opaque to X-rays and disposed relative to one another in groups of three to define two substantially parallel planes (P₁, P₂).

15. A system according to claim 12, characterized by the fact that making the fifth image by raster analysis using radiation that is absorbed relatively little by flesh and that is suitable for being reflected by the portion of bone comprises means for determining at least three points associated with said portion of bone, and means for defining, relative to the first reference (R₁) a reference surface in three dimensions as a function of said at least three points associated with said portion of bone.

16. A system according to claim 15, characterized by the fact that the means for determining at least three points associated with said portion of bone comprise a depth probe suitable for emitting one of the following kinds of radiation: ultrasound; and coherent light.

17. A system according to claim 11, characterized by the fact that the first reference (R₁) (FIG. 2) is constituted by at least four points (141, 142, 143, 144) of brightness contrasting relative to the brightness of the surroundings, one of said four points (144) being situated outside the plane (P₃) defined by the other three points (141, 142, 143).

18. A system according to claim 11, characterized by the fact that the reference (R₂) (FIG. 4) is constituted by a plurality of points (151, 152, 153, . . . , 161, 162, 163) of brightness contrasting relative to the brightness of the surroundings, said points being associated with a said tool (3) in such a manner that at least three of said points are always visible from a location in the space in which said tool is suitable for being displaced and regardless of the position of said tool (3) relative to said first reference (R₁).

19. A system according to claim 18, characterized by the fact that plurality of points (151, 152, 153, . . . , 161, 162, 163) of contrasting brightness associated with said tool (3) are distributed around said tool (3) in substantially regular manner in two planes (P₄, P₅) that are substantially mutually parallel and that do not coincide, each of said two planes (P₄, P₅) including at least three of said points (151, 152, 153, . . . , 161, 162, 163) of contrasting brightness associated with said tool.

20. A system according to claim 11, characterized by the fact that the means (20) for making the third image are constituted by:
processor means (122) suitable for processing said first and second images to transform them into digital images and for superposing them to give a final digital image; and
means (123) for converting said final digital image into an optical image constituting said third image.

21. A system according to claim 20, characterized by the fact that said means (30) for projecting the third image into the field of view (4) of an eye of the practitioner (1) while the practitioner is ready to manipulate said tool (3) are constituted by means (127) for focusing said third image outside said field of view (4), together with a semireflective plate (128) for causing the optical axis of said focusing means (127) to coincide optically with the axis of said field of view (4) to deliver said third image into the practitioner's eye.

22. A system according to claim 14, characterized by the fact that the means (11) for making a fourth image by stereovision comprise two video cameras (111, 112), a transverse support (113), and means for mounting said two video cameras respectively substantially at the two ends of said support so that their optical axes coincide.
